(19) United States
(12) Patent Application Publication
(10) Pub. No.: US 2019/0343907 A1
(43) Pub. Date: Nov. 14, 2019

(54) Synergistic Compositions and Methods of Achieving Homeostasis in Mammalian Systems

(71) Applicant: WhiteHill Life Sciences, LLC, Stafford, TX (US)

(72) Inventors: Dale G. Brown, Wharton, TX (US); Ira D. Hill, Austin, TX (US)

(21) Appl. No.: 16/408,504

(22) Filed: May 10, 2019

Related U.S. Application Data
(63) Continuation of application No. PCT/US18/34296, filed on May 24, 2018.
(60) Provisional application No. 62/522,336, filed on Jun. 20, 2017.

(51) Int. Cl. A61K 36/18; A61K 31/16

(52) U.S. Cl. CPC A61K 36/18; A61K 31/16

(57) ABSTRACT

In one aspect, this invention relates to a composition of matter useful for treating homeostatic malfunction and subsequent resulting conditions. In another aspect, this invention relates to the disclosure of unexpected synergies created by certain mixtures that increase the nature and function of those mixtures over any of the ingredients alone. In another aspect, this invention relates to a method for treating homeostatic instability conditions. In yet another aspect, this invention relates to formulating a synergistic agent to return mammalian systems to normal set point conditions—i.e., homeostasis—across a surprising number of different pain, inflammation and other expressions of non-homeostasis.

### CROSS-REFERENCE TO RELATED APPLICATIONS

This application is a continuation of commonly owned PCT Application No. PCT/US18/34296, filed 24 May 2018. The PCT Application claims domestic priority under 35 U.S.C. 119(e) to commonly owned U.S. Provisional Application Ser. No. 62/522,336, filed Jun. 20, 2017. The disclosures of these applications are hereby incorporated herein by reference.

### FIELD OF THE INVENTION

In one aspect, this invention relates to a composition of matter useful for treating homeostatic malfunction and subsequent resulting conditions. In another aspect, this invention relates to a method for treating homeostatic instability conditions. In yet another aspect, this invention relates to formulating an agent to return mammalian systems to normal set point conditions, i.e., homeostasis.

### BACKGROUND OF THE INVENTION

The physical body of mammals utilizes control pathways for cell division, differentiation, wound repair, senescence; bacterial, viral and fungal infections; cancer and brain memory functionality, and essentially all mammalian metabolism and bodily functions. Control of homeostasis is a necessary path to the integrity of a mammalian body. (Kotas and Medzhitov, Cell 160, pg. 816-827, 2015).

Homeostatic control circuits have set points, regulated variables, control variables and controller gain (amplified response rate). Perturbation of mammalian systems results in a bodily response in an attempt to return the system to the set point. These perturbations may take the form of uncontrolled growth (cancer), uncontrolled energy substrates (diabetes), loss of memory control (Alzheimer's), wounds, itching, viral infections, bacterial infections, fungal infections, immune dis-regulation (lupus, Sjogren's, Crohn's disease, irritable bowel disease), blood pressure deviation, acute inflammation, chronic inflammation, burns (x-rays, sunburn, thermal burns), aging cells, loss of hair follicle production, joint inflammation (rheumatoid arthritis, osteoarthritis), psoriasis, eczema, atopic dermatitis, mucositis, Aphthous ulcers, rosacea, and the like.

Loss of homeostatic control to a mammalian system can have catastrophic results, i.e., death, but in most instances, it results in an attempt to return the mammalian system to the particular set point. Areas of immediate control are oxygenation levels in the blood; nerve conduction in control of muscles, i.e., breathing; brain processing of feedback signals from touch, sight and sound.

These acute systems require a quantitative sensor; a feedback mechanism for responding to the signal. Ion channels that control sodium, potassium and calcium ions entry to the cellular cytoplasm are of major importance to carrying out the function of the particular organ under study. Modulation of the acute system to external inputs allows a many-fold response to touch, pain, heat, pH and diseases states.

Restoring homeostasis to the set point is accomplished by healing mechanisms through responding to acute and chronic set points.

### SUMMARY OF THE INVENTION

In one aspect of the invention, there are provided synergistic compositions whereby homeostasis of a mammalian system, in need of returning to the set point, i.e., a healthy state, is achieved by administration of the composition to a patient in need of such treatment.

In another aspect of the invention, there are provided methods for returning a mammal to a homeostatic set point, i.e., a healthy state, by administration of a synergistic composition to a patient in need of such treatment.

In accordance with another aspect of this invention there are provided methods to treat a mammal with synergistic compositions to return to the homeostatic set point.

Mammalian homeostatic dis-regulations, such as: oral pain, joint pain, diabetic wounds, high blood pressure, and stem cell functionality, cold sores, thrush, shingles, Alzheimer's disease, cancer, radiation dermatitis, burns, Crohn's disease, psoriasis, scar formation, inflammation, pruritus, Sjogren's Syndrome, insect bites and stings (with or without accompanying anaphylactic shock) and various other allergic responses to foods, pollens, environmental allergens, etc., are treated with synergistic compositions to help return the mammal to the respective homeostatic set point.

### DEFINITIONS

As used herein, HOMEOSTASIS is defined as: The tendency of a system, especially the physiological system of higher animals, to maintain internal stability, owing to the coordinated response of it parts to any situation or stimulus that would tend to disturb its normal conditions or function. See, The American Heritage® Science Dictionary. Also, “a tendency toward maintenance of a relatively stable internal environment in the bodies of higher animals through a series of interacting physiological processes.” See, Merriam Webster Unabridged Online.

As used herein, SYNERGY is defined as the effects of combined components interacting to produce the effects of combined components interacting to produce, increased or new and different effects than the individual components. Typically used to refer to the action of whole organisms, as opposed to active constituents in isolation. See, Multiple Specialized Dictionaries in Biology and Medical terms.

### EMBODIMENTS OF THE INVENTION

As described herein, one embodiment of the invention is a synergistic homeostatic composition comprising one or more vitamins combined with one or more additive components selected from the group consisting of natural herbal extracts, and nature-identical synthetic chemical entities whose structures are active in restoring homeostasis to a mammal, vitamins and surfactants increasing the effect of the entire mixture. This composition may include a pharmaceutically acceptable carrier which may or may not include the activity increasing surfactant.

In an embodiment of the invention, a pharmaceutically acceptable carrier is formulated with a surfactant which significantly improves saliva flow and effects similar bodily functions better than other surfactants. Preferred nonionic surfactants that can be formulated into a pharmaceutically acceptable carrier having these characteristics are; BASF PLURACARE® F 127, BASF PLURACARE® F 108, BASF PLURACARE® L-1220, INCI-named PEG/PPG-116/66 Copolymer and PLURACARE® L-4370 INCI-named PEG/PPG-38/8 Copolymer, either alone or together.

As described herein, one embodiment of the invention is a method of treating a subject in need of homeostasis by administering to a subject in need of such treatment, a synergistic homeostatic amount of a composition comprising one or more vitamins combined with one or more additive components selected from the group consisting of natural herbal extracts, and nature-identical synthetic chemical entities whose structures are active toward restoring homeostasis. In this method, the synergistic composition may further include an OTC Drug ingredient, or an approved Prescription Drug, thereby providing improved homeostatic performance of the Drug.

The OTC Drug ingredient may be selected from the groups, subgroups and ingredients listed in the FDA's 21 C.F.R. list of approved OTC drugs ranging alphabetically from Acne to Wart Removal. OTC drugs are defined by the FDA as “drugs that are safe and effective for use by the general public without seeking treatment by a health professional.” OTC monographs define the safety, effectiveness, and labeling of OTC active ingredients. The Prescription Drug list and approved claims may be accessed online at Drugs@FDA.

In any combination with either OTC or Rx Drugs, the synergistic, homeostatic mixtures are not drugs per se, rather they serve as excipients whose purpose is to enable the chosen drug to maximize its efficacy by encouraging the homeostatic balance and relief from symptoms accompanying the intended use of the drug.

Given the complexity of drug mechanism of action, not all of either the OTC or Rx list can be expected to exhibit synergy and/or enhanced homeostasis. However, once the synergistic and homeostatic principles of this patent are understood, using the claimed ingredients or classes described in this patent, along with described methods of formulation, method of application and utility resulting therefrom; anyone skilled in the art can routinely examine any desired Drug's synergistic or homeostatic response to a combination of the Drug with the mixtures and art herein described.

In a method of the present invention, the homeostatic malfunction condition may be caused by the entire spectrum of malfunctions, related to a body condition selected from the group consisting of, but not limited to: chronic and acute pain, hair loss, ageing, Alzheimer's disease, diabetes, arteriosclerosis, actinic keratosis, radiation dermatitis, pruritus, insect bites, allergic responses to allergens in pollens, foods, personal care products and chemical ingredients, insect stings (up to and including anaphylactic shock), carbuncles, dermal burns due to exposure to sun, x-rays and/or excessive heat, cancer, Sjogren's Syndrome, mucositis, aphthous ulcers, periodontal and gingivitis disease, viral infections, yeast and/or microbial infections, psoriasis, Crohn's disease, lupus, irritable bowel disease, other autoimmune diseases and diseases causally related to autoimmune diseases like fibromyalgia, bruises, wound healing and scar formation (whether accidental or surgical), rheumatoid and osteoarthritis, and xerostomia, blood pressure elevation, and the like.

In a method of the present invention, the composition may be administered to the subject by a route selected from the group consisting of mucosal, dermal, oral, anal, inhalation, injection, and other common routes known in the medical arts. In the method, the synergistic composition further may include one or more herbal extracts and/or nature-identical synthetic alkylamides and other molecules therein which are effective for achieving homeostasis. In the method, the alkylamides may be selected from the group consisting of spilanthol, pellitorine, alpha-hydroxysanshool, and mixtures thereof.

In a method of the present invention, the herbal extracts and/or nature-identical synthetic chemical entities may be selected from the group consisting of: Corydalis yanhusuo, Scopolia carniolica, Asperula odorata, Salix alba, Aloysia citrodora, Rhodiola rosea, Raphanus sativus, Pogostemon patchouli, Paeonia alba, Thymus mastichina, Magnolia biondii, Ligusticum chuanxiong, Lavandula angustifolia, Cypripedium reginae, Ginko biloba, Gelsemium sempervirens, Tenacetum parthenium, Piscidia erythrina, Harpagophytum procumbens, Salvia sclerea, Nepta cataria, Petasites hybridus, Iris versicolor, Stachys betonica, Melissa officinalis, Ruta graveolens, Juniperus sabina, Hypericum perforatum, Anemone sylvestris, Pulsatilla vulgaris, Ranunculus gramineus, Delphinium staphisagria, Thuja occidentialis, Gaultheria procumbens, Pinus sylvestris, Mentha piperita, Schinus molle, Piper nigrum, Anacyclus pyrethrum, Urtica dioica, Moringa oleifera, Hyoscyamus niger, Conium maculatum, Allium sativum, Abies alba, Angelica sinensis, Croton tiglium, Juniperus virginiana, Capsicum annuum, Cinnamomum camphora, Acorns calamus, Melaleuca cajeputi, Dorema ammoniacum, Aconitum napellus, Illicium verum, Ferula foetida, Elettaria cardamomum, Sygyzium aromaticum, Trigonella foenum-graecum, Zingiber officinale, Solidago virgaurea, Armoracia rusticana, Glycyrrhiza glabra, Brassica nigra, Myristica fragrans, Allium sepa, Origanum vulgare, Zanthoxylum clava-herculis, Santalum album, Ulmas rubra, Eriodictyon californicum, Aerva lanata, Prunus serotine, Juniperus communis, Guaiacum officinale, Salvia officinalis, Malpighia glabra, Matricaria recutita, Eucalyptus globulus, Conyza Canadensis, Polygonum aviculare, Malva sylvestris, Aloe barbadensis, Origanum majorana, Sorbus Americana, Azadirachta indaca, Spilanthes acmella, Juglans regia, Melaluca alternifolia, Xanthorhiza simplicissima, Withania somnifera, Atropa belladonna, Valeriana officinalis, Morella cerifera, Syzygium cumini, Hypericum perforatum, Hamamelis virginiana, Rubrus fruticosus, Juglans cinerea, Bellis perennis, Gardenia augusta, Solidago virgaurea, Cetraria islandica, Avena sativa, Vinca minor, Santalum album, Polygonatum odoratum, Rhus aromatica, Vaccinium myrtillus, Trifolium pratense, Artium Lappa, Argentina anserina, Astragalus membranaceus, Paeonia lactaflfora, Baccharis trimera, Sanguinaria canadensis, Calendula officinalis, Murraya koenigii, Rumex crispus, Boswellia sacra, Baptisia tinctoria, Dryas octapetala, Parietaria officinalis, Arnica montana, Tanacetum vulgare, Echinacea angustifolia, Melilotus officinalis, Nymphaea alba, Aristolochia serpentaria, Hepatica nobilis, Rosmarinus officinalis, Symplocarpus foetidus, Gelsemium sempervirens, Phytolacca Americana, Staphania tetrandra, Melissa officinalis and Sterculia urens.

In a method of the present invention, the composition may be administered to the subject prior to, immediately after, and/or long-after the homeostatic malfunction condition develops.

In a method of the present invention, the inflammatory conditions are controlled by affecting one or more biochemical control systems selected from the group consisting of NRF2, NFkB, PI3K, AKT, MAPK, JNK, PIP2, PIP3, ERK1/2, cAMP, Adenylyl cyclase, Annexin A1, TRPV1, TRPA1, TRPM8, PGE2, TNFa, IL-1b, CXCR4, CXCL 12.

Although not wishing to be bound by the above mode of action, it is consistent with the human, animal and in-vitro case history results presented herein as UTILITY EXAMPLES, which are adequate to encourage the use of the described mixtures use for the desired relief of pain, inflammation and resultant resistance to healing from all sources.

In a method of the present invention, the herbal extracts and individual alkylamides may be administered in an amount from 0.01 mg to 1000 mg per dose. In certain embodiments of the method, the alkylamides may be administered in an amount from 0.1 mg to 500 mg per dose. One or multiple doses may be required on a daily basis for effective treatment.

In a method of the present invention, the vitamin may be selected from the group consisting of vitamins A, B, C, D, E, their pro-vitamin precursors, and mixtures thereof. In a preferred method, the vitamin may be pro-vitamin B5. In a preferred method, the vitamin may be administered at from 1.5 mg to 1000 mg per dose. In a more preferred method, the vitamin amount administered may be from 2.5 to 500 mg per dose. In a preferred method, the vitamin may be D3. In a preferred method, the vitamin amount administered may be from 400 IU to 10,000 IU per dose. In certain embodiments of the method, the vitamin amount administered is from 1000 IU to 5,000 IU per dose. One or multiple doses may be required on a daily basis for effective treatment.

As used herein, “SYNERGY” is defined as the unexpected effect of multiple ingredients of natural or nature-identical chemicals and vitamins, either as mixture of such ingredients alone, or combined with a wide range of drugs, both OTC and Rx, in achieving homeostasis of the body and thereby providing physical relief of the intensity, duration, onset, and alleviation of various conditions of the body by (1) producing temporary relief of discomfort, pain, and inflammation, (2) improving or modifying bodily function, up to (3) providing permanence of physical relief and/or cure of various disease states of the body.

It should be noted that the literature (both folk and scientific) is replete with examples of individual natural extracts (and by extension—nature-identical molecules produced synthetically) benefitting various problematic pain and infection symptoms. However, Applicant submits that the present invention, namely a Synergistic, Homeostatic Mixture of Ingredients, including vitamins and their precursors, which simultaneously and positively impacts a wide range of Pain, Inflammation, Auto-Immune, Allergic Response, Radiation, and like diseases and conditions, with both rapid and extensive relief, is both an unexpected and surprising discovery.

Preferred embodiments of the invention include the following:
1. A synergistic homeostatic composition comprising one or more vitamins combined with one or more additive components selected from the group consisting of natural herbal extracts, and nature-identical synthetic chemical entities whose structures are active in restoring homeostasis to a mammal.
2. The composition of Embodiment 1, further including a pharmaceutically acceptable carrier.

3. The composition of Embodiment 2, wherein the pharmaceutically acceptable carrier further includes a surfactant.
4. The composition of Embodiment 3, wherein the surfactant is selected from L-1220, L-4370, or a combination thereof.
5. A method of treating a subject in need of homeostasis by administering to a subject in need of such treatment, a synergistic, homeostatic amount of a composition comprising one or more vitamins combined with one or more additive components selected from the group consisting of natural herbal extracts, and nature-identical synthetic chemical entities whose structures are active toward restoring homeostasis.
6. The method of Embodiment 5, wherein the composition further includes a pharmaceutically acceptable carrier.
7. The method of Embodiment 6, wherein the pharmaceutically acceptable carrier further includes a surfactant.
8. The method of Embodiment 7, wherein the surfactant is selected from L-1220, L-4370, or a combination thereof.
9. The method of Embodiment 5, where the synergistic composition further includes an OTC Drug and/or an Rx Drug ingredient, thereby providing synergy and improved homeostatic performance of the OTC or Prescription Drug.
10. The method of Embodiment 9, wherein the OTC or Rx Drug ingredient is selected from the Approved Group listed in FDA 21 CFR.
11. The method of Embodiment 5, wherein the homeostatic malfunction condition is caused by or related to a body condition selected from the group consisting of: Chronic and Acute Pain of the muscles, joints and ligaments, Migraine Headaches, Hair Loss, Thin, fragile skin, transparency and surface flaws which accompany Ageing, Alzheimer's disease, Diabetes, Arteriosclerosis, Actinic keratosis, Radiation Dermatitis, Pruritus, Insect bites, Anaphylactic Shock from serious allergic responses to allergens from Insect Bites, Food Allergies, Pollens and other Environmentally induced allergies, Carbuncles, Dermal burns due to exposure to sun, x-rays and/or excessive heat, Cancer, Mucositis, Aphthous ulcers, Periodontal and Gingivitis disease, Viral infections, Yeast and/or Microbial infections, Psoriasis, Crohn's disease, Lupus, Irritable Bowel disease, other Autoimmune Diseases and Diseases causally related to autoimmune diseases like fibromyalgia, Bruises, Wound Healing and Scar Formation (whether accidental or surgical), Rheumatoid and Osteoarthritis, multiple intensities of Dry Mouth or Xerostomia, and Dry Eyes.
12. The method of Embodiment 5, wherein said composition is administered to the subject by a route selected from the group consisting of mucosal, dermal, oral, inhalation, injection, anal, nasal and other common routes known in the medical arts.
13. The method of Embodiment 5, wherein the synergistic composition further includes one or more herbal extract and/or nature-identical synthetic alkylamides effective for achieving homeostasis.
14. The method of Embodiment 13, where the alkylamides are selected from the group consisting of spilanthol, pellitorine, alpha-hydroxysanshool, and mixtures thereof.
15. The method of Embodiment 5, wherein the herbal extracts and/or nature-identical synthetic chemical entities are selected from the group consisting of: Corydalis yanhusuo, Scopolia carniolica, Asperula odorata, Salix alba, Aloysia citrodora, Rhodiola rosea, Raphanus sativus, Pogostemon patchouli, Paeonia alba, Thymus mastichina, Magnolia biondii, Ligusticum chuanxiong, Lavandula angustifolia, Cypripedium reginae, Ginko biloba, Gelsemium sempervirens, Tenacetum parthenium, Piscidia erythrina, Harpagophytum procumbens, Salvia sclerea, Nepta cataria, Petasites hybridus, Iris versicolor, Stachys betonica, Melissa officinalis, Ruta graveolens, Juniperus sabina, Hypericum perforatum, Anemone sylvestris, Pulsatilla vulgaris, Ranunculus gramineus, Delphinium staphisagria, Thuja occidentialis, Gaultheria procumbens, Pinus sylvestris, Mentha piperita, Schinus molle, Piper nigrum, Anacyclus pyrethrum, Urtica dioica, Moringa oleifera, Hyoscyamus niger, Conium maculatum, Allium sativum, Abies alba, Angelica sinensis, Croton tiglium, Juniperus virginiana, Capsicum annuum, Cinnamomum camphora, Acorns calamus, Melaleuca cajeputi, Dorema ammoniacum, Aconitum napellus, Illicium verum, Ferula foetida, Elettaria cardamomum, Sygyzium aromaticum, Trigonella foenum-graecum, Zingiber officinale, Solidago virgaurea, Armoracia rusticana, Glycyrrhiza glabra, Brassica nigra, Myristica fragrans, Allium sepa, Origanum vulgare, Zanthoxylum clava-herculis, Santalum album, Ulmas rubra, Eriodictyon californicum, Aerva lanata, Prunus serotine, Juniperus communis, Guaiacum officinale, Salvia officinalis, Malpighia glabra, Matricaria recutita, Eucalyptus globulus, Conyza Canadensis, Polygonum aviculare, Malva sylvestris, Aloe barbadensis, Origanum majorana, Sorbus Americana, Azadirachta indaca, Spilanthes acmella, Juglans regia, Melaluca alternifolia, Xanthorhiza simplicissima, Withania somnifera, Atropa belladonna, Valeriana officinalis, Morella cerifera, Syzygium cumini, Hypericum perforatum, Hamamelis virginiana, Rubrus fruticosus, Juglans cinerea, Bellis perennis, Gardenia augusta, Solidago virgaurea, Cetraria islandica, Avena sativa, Vinca minor, Santalum album, Polygonatum odoratum, Rhus aromatica, Vaccinium myrtillus, Trifolium pratense, Artium Lappa, Argentina anserina, Astragalus membranaceus, Paeonia lactaflfora, Baccharis trimera, Sanguinaria canadensis, Calendula officinalis, Murraya koenigii, Rumex crispus, Boswellia sacra, Baptisia tinctoria, Dryas octapetala, Parietaria officinalis, Arnica montana, Tanacetum vulgare, Echinacea angustifolia, Melilotus officinalis, Nymphaea alba, Aristolochia serpentaria, Hepatica nobilis, Rosmarinus officinalis, Symplocarpus foetidus, Gelsemium sempervirens, Phytolacca Americana, Staphania tetrandra, Melissa officinalis and Sterculia urens.
16. The method of Embodiment 5, wherein the composition is administered to the subject prior to, immediately after, and/or long-after the homeostatic malfunction condition develops.
17. The method of Embodiment 5, wherein the vitamin is selected from the group consisting of vitamins A, B, C, D, E, their pro-vitamin precursors, and mixtures thereof 18. The method of Embodiment 5, wherein the inflammatory conditions are controlled by affecting one or more biochemical control systems selected from the group consisting of Annexin A1, NRF2, NFkB, PI3K, AKT, MAPK, JNK, PIP2, PIP3, ERK1/2, cAMP, Adenylyl cyclase, TRPV1, TRPA1, TRPM8, PGE2, IL-1b, CXCR4 and CXCL 12
19. The method of Embodiment 5, wherein the inflammatory condition is reduced and homeostasis is restored by marshalling or enhancing the activity of the stem cells resident in the mammal by one or more of the stem cells' primary functions, (a) cell renewal and reproduction, (b) expressing chemotaxis allowing the stem cells to move toward a signal indicating an area of inflammatory condition, (c) cellular differentiation to produce the type of cell needed for reconstruction and (d) by reducing TNFa and increasing IL10, thus allowing a return to homeostasis.
20. The method of Embodiment 13, wherein the alkylamides are administered in an amount from 0.1 mg to 1000 mg per dose.
21. The method of Embodiment 13, wherein the alkylamides are administered in an amount from 0.1 mg to 500 mg per dose.
22. The method of Embodiment 17, wherein the vitamin is pro-vitamin B5.
23. The method of Embodiment 21, wherein the vitamin is administered at from 2.5 mg to 1000 mg per dose.
24. The method of Embodiment 22, wherein the vitamin is administered at from 2.5 to 500 mg per dose.
25. The method of Embodiment 17, wherein the vitamin is D3.
26. The method of Embodiment 25, wherein the vitamin is administered at from 400 IU to 10,000 IU per dose.
27. The method of Embodiment 25, wherein the vitamin is administered at from 1000 IU to 5,000 IU per dose.

It will be appreciated by those persons having ordinary skill in the art(s) to which the present invention relates that any of the features described herein in respect of any particular embodiment and/or embodiment of the present invention can be combined with one or more of any of the other features of any other embodiments and/or embodiments of the present invention described herein, with modifications as appropriate to ensure compatibility of the combinations. Such combinations are considered to be part of the present invention contemplated by this disclosure.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

### DETAILED DESCRIPTION OF THE PREFERRED EMBODIMENTS

Pain is a result of a homeostatic dis-regulation and it constitutes an “unpleasant sensory and emotional experience associated with actual or potential tissue damage”, (Mickle et al., Pharmaceuticals, 2016, 9, article 72). Pain acts as an alarm system and a protective mechanism for a wide range of homeostatic dis-regulatory conditions, i.e., pathological conditions.

The first and foremost pain process is the peripheral detection and transduction of noxious stimuli that are determined as painful by the higher-order structures in the central nervous system. The terminology that has been widely used to define this process is “nociception”, which accounts for the neural mechanisms and pathways for the encoding and processing of noxious stimuli.

Transient Receptor Channels (TRP), are ion channels used as sensory detectors and transducers of many pain pathologies, for example, inflammatory pain, dental pain, migraine, neuropathic pain, visceral pain, heat, acid, mechanical and temperature.

More specifically, calcium ion channels that control pain are TRPV1, TRPA1 and TRPM8. Antagonists and agonists act to sensitize or tamp down the response to pain. (Bourinet et al., Physiol. Rev. 2014, 94, 81-140).

Multiple control sites in the TRPA1 ion channel proteins allow modulation for high and low states of response to pain stimuli. (Paulsen et al., Nature, 2015, 520, 511-517).

Co-expression of TRPV1-A1 complexes in dorsal root ganglia (DRG) have been found to be regulated by the connector Tmem100 in the instance of complex and persistent pain. (Weng et al., Neuron, 2015, 85, 833-846).

The arachidonic pathway pain modulators have a major influence on the sensation of pain. Some endogenous inhibitors of pain have been shown to inhibit both TRPV1 and TRPA1 with potent pain relief activity. (Park et al., J. Neuroscience, 2011, 30 (50), 18433-18438).

From the above references, a suitable pain relief composition may be derived from those structures that affect pain by dermal and mucosal transport, or by oral ingestion and anal application. The dual ion channel approach would seem to be productive in relieving pain.

A synergistic pain relief composition may combine multiple homeostatic control mechanisms to be effective. Suitable alkylamides and natural extracts with known ion channel and pain relief properties combined with other biochemical modifiers such as vitamins or their precursors has been found to exert pain relief rapidly and for extended periods of time.

Spilanthes species have reported pain relief properties in many review articles. Some species are called the “toothache plant”. Paulraj et al., Advances in Pharmacological Sciences, Volume 2013, article ID 510298, details worldwide use of the genus in traditional medicines for pain, cancer, cough, xerostomia, mouth ulcers, wound healing, infections, eczema, local anesthetic and rheumatism,

It has been found that alkylamides in herbal extracts or synthetic equivalents in combination with pro-vitamin B5 formulated in a suitable carrier give dermal and mucosal pain relief of a rapid nature. For example, sore throats and oral sores such as canker sores, cold sore/fever blisters, thrush and other infections by various viruses, bacteria and yeasts may be treated with sprays, rinses, lozenges, gels and toothpastes to deliver transmucosal relief.

The ability of alkylamides to penetrate the skin and to enhance the penetration of other chemical structures has been demonstrated. (Boonen et al., J. Ethnopharma. 2010, 127, 77-84) and (Veryser et al., Skin Pharmacol. Physiol. 2015, 28, 124-136).

The primary pain-relieving agent in Spilanthes extracts is spilanthol, an isobutyl substituted unsaturated olefinic amide. Historically, extracts such as black pepper oil containing trans-pellitorine and other alkylamides work synergistically with the major components of the extracts to hasten and strengthen the achievement of homeostasis. The pain-relieving action is also accompanied by an anti-inflammatory activity operating through the NFkB inflammatory pathway. (Wu et al., J. Ag. Food Chem., 2008, 56, 2341-2349).

The inflammatory pathways connected to NFkB regulate cancer, diabetes type 2, Alzheimer's, Sjogren's syndrome, wound healing, radiation dermatitis, rheumatoid arthritis, osteoarthritis, cold sores, shingles and aging.

Testing of spilanthese extract and spilanthol and/or other extract ingredients such as pellitorine, in oral and dermal vehicles has shown a return to homeostasis. In acute pain, a rapid reduction in inflammation and pain often occurs within minutes or hours; in chronic inflammation, positive results after 2-3 weeks of oral or dermal applications.

These wide-ranging positive effects point to a common homeostatic mechanism, which returns the organism to the set point by marshalling or enhancing the stem cells activity of the stem cells resident in the mammal. This allows, per se, the body to participate in its own achievement of homeostasis, whether for relief of temporary symptoms or permanent healing. This, in turn, is “turned on” by the synergistic, homeostatic mixture as demonstrated in the Utility Examples from the Case Histories of the mixture's demonstrated effect on multiple expressions of the loss of homeostasis.

The homeostatic condition utilizes stem cells which interact with the aforementioned biochemical control systems. Although not bound by this mechanism, the control of pain, inflammatory conditions and wound healing is consistent with actions on TGF-beta, NFkB, PI3K, AKT, PIP2, PIP3, IKKbeta, TRPM8, TRPV1, TRPA1, Annexin A1, CXCR4, CXCL 12 and AcetylCoA. (Loo et al., Pain Journal, 2015, 156, S1-S10) and (Gilbert et al., J. Develop. Biol., 2016, 4, (2), 21).

Formulations of spilanthol and/or spilanthes extract, and/or other extracts suitable for oral use range from 0.0025 wt % to 0.4 wt % and the pro-vitamin B5 from 0.5 wt % to 2 wt % for relief from oral pain due to cold sores, mucositis, aphthous ulcers, gingivitis, periodontitis, thrush and sore throat. Pellitorine may also be included at 0.025 wt % to 0.6 wt %.

Formulations for skin treatment include spilanthol and/or spilanthes extract at 0.4 wt % to 6 wt % in a vehicle compatible with pro-vitamin B5.

Formulations for joint pain should include spilanthol and/or spilanthes extract, and/or other extracts containing active ingredients such as alkylamides at 0.4 wt % to 5 wt % along with pro-vitamin B5. Capsaicin skin creams are enhanced in pain relief and in speed of relief by the addition of spilanthol and/or spilanthes extract with along with menthol or coolants WS3, WS23, or WS-5.

### FORMULATION EXAMPLES

### Example 1

### Skin Cream Lotion

A 500 mL stainless steel beaker fitted with an overhead stirrer was placed on a hot plate with 141.28 gm of water. The heat was adjusted to 85° C. with moderate stirring while 0.02 gm of sodium chloride was added. Colloidal oat kernel flour, 2.0 gm was added to the beaker with continued stirring for 5 minutes. Glycerin, 24 gm, was added followed by distearyldimonium chloride, 10 gm, white petrolatum, 8 gm, polydimethylsiloxane, 350 centistokes (CS), 2.5 gm, cetyl alcohol, 5 gm and isopropyl palmitate, 6 gm, all with stirring for 10 minutes at the 85° C. temperature. The beaker was allowed to cool to 40° C. and benzyl alcohol, 1.2 gm was added with continued stirring. After cooling to 30° C. over 5 minutes, pro-vitamin B5, 12 gm, and spilanthes extract, 3.68 gm were added with slow stirring until cooled to 25° C. The thick lotion was added to plastic tubes, which were suitable for dispensing onto skin.

### Example 2

### Oral Gel

A one Liter stainless steel beaker (tank A) was fitted with an overhead stirrer. Water, 266.265 gm, was added and moderate stirring began. Additional ingredients for this vessel were added: Sorbitol 70%, 204 gm; glycerin, 30 gm; potassium sorbate, 0.9 gm; sodium saccharin, 0.45 gm; sucralose, 1.2 gm; mint flavor, 2.0 gm; Spilanthes extract, 0.48 gm and Spilanthol, 0.3 gm; pro-vitamin B5, 1.0 gm were added with moderate stirring at room temperature.

A 250 mL beaker (tank B) containing an aqueous-free emulsion [Pluracare® L-1220/polydimethylsiloxane (2.5 million CS)] (90:10) 19.08 gm, was heated to 95° C. with magnetic stirring.

To a 250 mL beaker (tank C), with overhead stirring and heating, was added propylene glycol, 60 gm, and methyl paraben, 0.9 gm. When the temperature attained 50° C., carboxymethylcellulose 9M31F, 15 gm, was added slowly over 3 minutes. After 5 minutes of stirring, the contents were added slowly to tank B, containing the emulsion. After continued stirring and cooling to 40° C., the contents of tank B were added to tank A slowly over 5 minutes. After an additional 30 minutes, the oral gel was packaged for dispensing from tubes to the oral cavity to stimulate saliva flow that relieved oral pain and give a pleasant mint tasting, tingling effect on the tongue.

### Example 3

### Joint Pain Cream

A 500 mL stainless steel beaker fitted with an overhead stirrer was placed on a hot plate with 141.28 gm of water. The heat was adjusted to 85° C. with moderate stirring while 0.02 gm of sodium chloride was added. Colloidal oat kernel flour, 2.0 gm was added to the beaker with continued stirring for 5 minutes. Glycerin, 24 gm, was added followed by distearyldimonium chloride, 10 gm, white petrolatum, 8 gm, polydimethylsiloxane, 50 CS, 2.5 gm, cetyl alcohol, 5 gm and isopropyl palmitate, 6 gm, all with stirring for 10 minutes at the 85° C. temperature. The beaker was allowed to cool to 40° C. and benzyl alcohol, 1.2 gm, was added with continued stirring. After cooling to 30° C. over 5 minutes, pro-vitamin B5, 12 gm, and spilanthes extract, 3.68 gm; menthol, 4.3 gm and capsaicin, 0.21 gm were added with slow stirring until cooled to 25° C. The thick lotion was added to plastic tubes, jars or containers fitted with a treatment pump, which were suitable for dispensing onto skin. Application to sore and swollen joints gave an initial cooling, then warming effect.

### Example 4

### Lip Balm

A 400 mL glass beaker was fitted with an overhead stirrer and placed on a hot plate. Pluracare L-1220, 22 gm was added and heated to 80° C. Dow Elastomer® 9041, 66 gm, was added along with PEG 400. After 5 minutes, shea butter, 30 gm, cocoa butter, 30 gm, glycerin, raspberry flavor, 0.4 gm, spilanthes extract, 1.2 gm and pro-vitamin B5, 0.4 gm, were added with stirring for 5 minutes. The overhead stirrer was removed and a Ross M/E100LC homogenizer was inserted. The gel was homogenized at 7000 rpm for 10 minutes. The gel was allowed to cool to room temperature and added to small dispensing tubes. The lip balm gave a smooth layer to the lips with a pleasant raspberry flavor. A pleasant, tingling effect was evident when licking the lips.

### Example 5

### Oral Compressed Tablet

A 4 quart Hobart mixer with oscillating spade blade was prepared for mixing the tablet formula. Isomalt (Beneo 720) 939.76 gm was added along with Pluracare® L-1220, 30 gm, raspberry flavor, 3.0 gm, spilanthes extract, 0.35 gm, pellitorine, 0.125 gm, magnesium stearate, 15 gm, sucralose, 1.65 gm, pro-vitamin B5, 5 gm and citric acid powder, 5 gm. The mixer was stirred on low speed for 15 minutes. The resulting powder was added to a tablet press to give 1.5 gm tablets that delivered a raspberry flavor along with a tingling and a good salivating result.

### ILLUSTRATIVE FORMULATION EXAMPLES

### Example 6

### Pain Relieving Nasal Spray

Water, 957 gm, is added into a stainless steel beaker with moderate overhead stirring. A separate glass beaker is stirred with a magnet with 10 gm of water, 22 gm of propylene glycol, 50 mg of spilanthol, 10 gm of dexpanthenol and 1 gm codeine phosphate. When the glass beaker solution becomes homogeneous, it is added to the stainless steel beaker with continued stirring for 20 minutes. The resulting clear solution is added to 2 OZ nasal spray bottles. Dispensing one dose in each nostril gives rapid pain relief with reduced habituation propensity.

### Example 7

### Anal Suppository for Pain Relief

Two thousand grams of a low melting fatty acid glyceride, such as Witepsol W 35, is melted in a stainless steel vessel with overhead stirring and heating to 50° C. D-panthenol, 100 gm, is added along with 10 gm of Spilanthes acmella extract and continued stirring for 15 minutes. The molten mass is poured into 2 gm molds and cooled for 30 minutes. The resulting anal wax suppositories are suitable for rapid pain relief.

### Example 8

### Lozenge

Hard cooked isomalt lozenges are prepared with a dry weight formula comprised of: isomalt STM, 94.32 wt %; water, 2.08 wt %; poloxamer 407, 1.21 wt %; polydimethylsiloxane 2.5 million CS, 0.303 wt %; malic acid, 0.52 wt %; flavor, 0.31 wt %; sucralose 0.108 wt %; Acesulfame K, 0.052 wt %; spilanthese extract, 0.035 wt %; Spilanthol 0.11 wt %, d-panthenol, 0.45 wt %, pellitorine, 0.15 wt % and FD&C Red No. 40. The lozenges have a tingling effect on the tongue and stimulate saliva flow, effectively moisturizing dry mouths and other uncomfortable or painful non-homeostatic conditions.

### Example 9

### Oral Conditioning Rinse

A Rinse functioning as an Oral Conditioner and providing Dry Mouth Relief is prepared with (wt %): Water (86.3135), PLURACARE® L1220 (1.6), polydimethylsiloxane 2.5 cs (0.4), d-panthenol (0.5), Na Saccharin (0.03), Sucralose (0.05), Xylitol (5), Erythritol (3), Glycerin (2), Sodium Benzoate (0.5), EDTA (0.05), Trans-pellitorine (0.01), Coolants WS 3 & 23 (0.005), Flavor (0.132) Takasago 151 (mix of Spilanthese Extract and Spilanthol) (0.0095), CMC (0.4). The resulting rinse soothes irritated oral mucosa, while stimulating saliva flow; effectively moisturizing dry mouths and other uncomfortable or painful non-homeostatic conditions.

### UTILITY EXAMPLES—BASED ON CASE HISTORIES

### Example A

A 71 year old male had a brown scaly lesion on the scalp diagnosed as actinic keratosis. Prior episodes of these were removed surgically. The lotion of Example 1 was supplied to the subject with directions to apply a pea-sized amount to the lesion twice daily. After three weeks, the lesion had disappeared.

### Example B

A 70 year old male had two brown scaly lesions on his forearm. The lotion of Example 1 was supplied to the subject with instructions to apply a small amount to the brown scaly lesions twice daily. After three weeks, the lesions had disappeared.

### Example C

An 80 year old male was suffering from neoplasia of the sternum due to excessive X-ray and CAT scan treatment two years prior. Red irritated, itchy skin was present on his sternum. The subject applied small amounts of lotion from Example 1 to the red area twice daily for 3 weeks. The irritation and itching ceased within 24 hours of application. The skin returned to normal color after 3 weeks. No further lotion was applied and the skin remained the normal color.

### Example D

An 80 year old male with a history of wrestling injuries had extreme pain and swelling in his shoulders upon rising in the morning. A cream of Example 1 was given to the subject with instructions to use twice daily and pre-treat the shoulders with a menthol cream five minutes before using the cream of Example 1. Mild heat was perceived. Within 3 days, pain and swelling began to diminish. After 3 weeks of applications, swelling and pain were gone. No further creams were applied and the symptoms did not return over the next several months.

### Example E

A 60 year old female had both knees damaged by rheumatoid arthritis. She walked slowly with a cane or walker. Twice daily applications of a cream of Example 1 to both knees over 3 weeks gave substantial relief and allowed the subject to walk without cane or walker.

### Example F

An 81 year old male had a history of cold sores (herpes). A tingling on the lip was a signal that an eruption was imminent. The subject applied the lotion of Example 1 to the tingling area on the lip at night. The next day no tingling was perceived. No eruption occurred and no redness or blisters appeared.

### Example G

A 60 year old female incurred breast cancer procedure requiring 15 rounds of chemo and radiation following surgery. After ten days following the last radiation treatment, the surgical scar remained red, itchy and prevented normal sleep. Application twice daily of the lotion of Example 1 to the red area gave relief of itching within 4 hours. After three weeks of twice-daily application to the red irradiated area, it had returned to normal looking skin. No further lotion was applied.

### Example H

A 70 year old male burned his finger with a soldering iron. The resulting blister was 3/8 inch long and 1/8 inch wide and tall. Pain was experienced from the burned area. Application of the lotion from Example 1 gave pain relief within 30 minutes. Twice daily application to the burned area for 7 days resulted in the skin of the blister flattening and re-attaching to the finger with no cracking or peeling of blister area.

### Example I

A 70 year old male experienced fire ant bites on his legs. Typical itching and burning was experienced for 30 minutes after being bitten. Application of the lotion of Example 1 gave pain relief in one hour. Twice daily application to the bite area returned the skin to normal with no redness and no pustule formation as normally experienced with fire ant bites.

### Example J

An 81 year old male experienced mosquito bites on his ankles and calves while on vacation. On returning to his hotel, he applied pea-sized amounts of the lotion of Example 1 to the bitten area about 1 hour after mosquito contacts. The itching was reduced very quickly, there was no further itching or redness as evidence. Subject reports that this experience was repeated throughout the mosquito season.

### Example K

An 81 year old male experienced a serious sunburn on his nose, having forgotten to apply sunscreen to his nose while in the sun all day. In the evening, a red and painful sunburn was visually evident, hot, and painful to touch on the nose. Application of a small amount of the lotion from Example 1 to the affected area on the nose resulted in immediate (˜30 sec) reduction in pain and temperature of the skin. The next morning the skin had lost its redness, was slightly tanned and was not sensitive to the touch. The skin never peeled off as expected.

### Example L

A 73 year old male was scheduled to have Mohs surgery on his scalp to remove a basal cell carcinoma. One week prior to surgery, the subject applied twice daily a pea-sized amount of the lotion from Example 1 to the area scheduled for surgery. After surgery and one week recovery followed by removal of the stitches, the application of the lotion of Example 1 was resumed. After three weeks of twice-daily application of the lotion, the incision area was returned to normal skin with no evidence of scar or surgical insult.

### Example M

A 71 year old female was diagnosed with shingles on the lower jaw and on the scalp. A prescription for acyclovir was taken but no relief from pain or itching was forthcoming within 1 week. The subject was given the lotion of Example 1 with instructions to pre-treat the area with a menthol cream five minutes before applying the treatment cream. Twice daily application to the border of the shingles lesions along the axis of red streaks prevented further expansion of the shingle lesions. In areas of blisters that had not ruptured, application of the treatment cream caused the blisters to recede and not rupture. After three weeks of twice-daily treatment, the redness and pain were substantially gone. Episodes of recurring post-herpetic neuralgia were treated successfully over the next six weeks with the cream giving reduction of redness, itching and pain.

### Example N

A 72 year old female had a history of bruising of the skin resulting in black areas turning to blue, followed by green a then yellow before disappearing over 3 weeks. A black bruise area on the arm and leg were treated with the lotion of Example 1 twice daily over one week. The treatment resulted in the blue color rapidly fading to light green and no color was observed after five days.

### Example O

A 35 year male singer, had to frequently sip water between songs. Placement on of one tablet weighing 1.5 grams, formulated as per Example 5, between the cheek and lower gums prior to beginning the session and allowing it to dissolve while singing, then another after 20 minutes allowed a full program to be completed without repeated sips of water on stage. The singer reported only slight intrusion of the physical presence of the tablet on enunciation.

### Example P

On his own volition, the singer of Example O applied a small amount of the oral gel from Example 2 around the rim of a water glass placed as usual within easy reach behind him onstage. The singer observed that unobtrusively touching his tongue to the gel just after a sip of water, and distributing the gel around his mouth with his tongue as he turned back to the audience, kept his oral cavity moisturized for about 6 songs. By repeating this application once or twice more, his full program could be sung with a moisturized mouth. This utility method had absolutely no intrusion on the enunciation or intonation of his singing.

### Example Q

A 60 year old female incurred breast cancer procedure requiring 15 rounds of chemo and radiation dermatitis after breast surgery. One week after last radiation treatment, the skin was red, scaly and itched. The itching prevented normal sleep patterns. Skin lotions and steroid creams did not help. Skin cream from Example 1 was applied in pea-sized amounts daily over 4 weeks to the surgical site beginning one week after last radiation treatment. Subject was able to sleep through the night on day one without itching. Redness was greatly diminished after three days. After four weeks, the skin had returned to normal color without any itching or redness.

### Example R

The female reported in EXAMPLE O (now 58 years old) reported that she was experiencing occasional psoriatic skin on the bridge of the nose. She was supplied with additional lotion of Example 1. She also was experiencing dry eyes which often accompanies dry mouth and took it upon herself to carefully apply small amounts of this lotion to areas around the eye and small amounts on the eye lid. She observed reduced dry eye symptoms. She continues to use the lozenges and skin cream on a daily basis.

### EXAMPLE S

A 50 year old male experienced stress-induced episodes of migraine headaches five to six times/year, each preceded by a “trigger signal” of slight dizziness or light-headedness. Sumatriptan prescription was slow to relieve the pain. The lotion of Example 1 was supplied by the subject. Application of pea-sized amounts of to the forehead and temples caused the trigger sensation to recede within 30 minutes and the migraine did not develop. No migraine episodes have occurred in the past eight months.

### Example T

A panel of 5 trained organoleptic evaluators evaluated the following properties of the Oral Conditioner Rinse of EXAMPLE 9. Results are Panel Averages: Scale of 1-5.

| Organoleptic | Initial Intensity | Time Perceived |
|---|---|---|
| Properties | (1-5) | (min) |
| PLEASANTNESS | 4.3 | 12 |
| CLEAN TEETH/MOUTH | 4.1 | 65 |
| SMOOTH MUCOSA | 4.5 | 57 |
| COMFORTABLE | 5.0 | 64 |
| MOISTURE | 4.8 | 37 |

### Example U

A 55 year old female nurse had suffered for many years with Crohn's disease, in which psoriasis on the knees and elbows occurred before each flare-up of abdominal pain. For many years her Crohn's incidence of abdominal pain, was sufficient to prevent frequent attendance at work. She was being treated by a gastroenterologist who found and removed the typical intestinal growths on multiple occasions. The frequency of flair-ups was about one to two times per month on a regular basis. Subject subsequently developed dry mouth and dry throat, to the extent that her voice began to fail, greatly interfering with her occupation as a teaching nurse. She was given lozenges of Example 8 to relieve dry mouth symptoms. The instant relief from Dry Mouth symptoms occurred with each lozenge. Over a three month period of using 4-5 lozenges per day, she remarked that the Crohn's symptoms seemed to diminish. And over the next 3 months her voice repaired sufficiently to return to daily lecturing. She reported that she had not had a Crohn's flair-up for the last three months. Upon inquiry about the frequency of psoriasis, she reported that they occasionally returned but were not followed by the dreaded flair-up. The psoriasis sites were still the typical; i.e., red, scaly, itching psoriasis sores on elbows and knees. She was provided with the lotion of Example 1 and began applying it to the sites twice daily as soon as she felt them starting to develop. The psoriasis development continued to occur, even though the pain of a Crohn's flare-up did not develop, but the lotion reduced the eruption to pink, non-scaly, non-itching, minor blemishes of short duration. At last report, she had experienced no Crohn's flare-up over the last 18 months.

### Example V

### Poison Ivy Relief Skin Cream

A male Lawn Maintenance Contractor in his late 50's got into a patch of Poison Ivy early in the morning while operating his Weed Whacker which threw bits of stem and leaves on his forearms and one spot on his forehead. By noon, at his next job site, he presented a serious itching problem and there were a number of individual “bumps” like he has experienced before all over his forearms and two on his forehead. There was also a “streak” on the underside of his forearm already broken out and red which likely came from the sap end of a stem cutting. He was given a skin cream (EXAMPLE 1) to rub generously over the itching areas, including those exposed and itching but had not yet produced a visible lesion. By the time he had washed his palms (to prevent accidently touching his eyes) the subject was saying amazedly “No More Itching! No More Itching”. There were no additional visible lesion after 3 hours when he finished the job. At noon the following day, the subject reported that the Poison Ivy lesions had not developed further and there was still no itching.

### ILLUSTRATIVE UTILITY EXAMPLES

### Example W

### Lip Balm

A 43 year old female residing in an arid climate exhibits both dry mouth symptoms and cracked lips. For one month she has been licking her lips with great frequency in an unconscious attempt to moisten her lips for clearer speech and comfort. Her lips are severely cracked and occasionally bleed. She uses a variety of OTC lip products, which give some immediate comfort, but do not succeed in reducing the number or depth of the cracks covering her upper and lower lips. She is given a tube of Formulation Example 5 and instructed to use as frequently throughout the day and night as she feels necessary, but at least once every two hours. The immediate result is a decrease in the frequency of involuntary licking of the lips. After three weeks of application according to the every two waking hours regimen, the cracks in her lips are no longer visible. Continuing to apply the lip balm at least twice a day and at bedtime, prevents a return of the cracked lips.

### Example X

### Pain and Stuffiness Relieving Nasal Spray

A male of approximately 80 years of age, chronically suffers from winter colds from which he is slow to recover. Thus for several months a season his nasal passages are very irritated and painful, and breathing is difficult. After one application of the Nasal Spray formulation of Example 7, at bedtime, easy breathing immediately returns and no pain or further stuffiness is experienced until the next day. The application is not accompanied by temporary irritation or pain of the nasal passage frequently experienced with commercial, OTC nasal sprays.

### Example Y

### Irritable Bowel Pain Relieving Anal Suppository

A 67 year old male has for many years experienced bouts of Irritable Bowel Syndrome at least once a month. Eight suppositories as described in Formulation Example 8, are provided and twice a day insertion is recommended. The subject complies and reports that the pain is greatly reduced 30 minutes after the first application and is gone completely after the second application of the day. The subject continues until all eight suppositories are used with no reoccurrence of irritable bowel pain during the 4 day experiment. Surprisingly, the subject experiences no reoccurrence of irritable bowels over the next four months.

### Example Z

### Gingival Healing with OTC Rinse and Oral Gel

A 31 year old male with serious gingival inflammation requiring periodontal surgery is first prescribed 10 days of twice-daily rinsing with a stable glycerin solution in Stannous Fluoride diluted to FDA standard concentrations for OTC use with a diluent consisting of the ingredients in Example 2. Major gingival surgery is performed and the subject receives an Oral Gel of Example 2 with instructions to rub the gel on all gingival surfaces, even those not subjected to surgery, on arise.

From the foregoing, it will be appreciated that although specific examples have been described herein for purposes of illustration, various modifications, obvious to one skilled in the art, may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

All references and other documents cited above are hereby incorporated herein by reference in their entirety.

What is claimed is:

1. A synergistic homeostatic composition comprising one or more vitamins combined with one or more additive components selected from the group consisting of natural herbal extracts, and nature-identical synthetic chemical entities whose structures are active in restoring homeostasis to a mammal.

2. The composition of claim 1, further including a pharmaceutically acceptable carrier.

3. The composition of claim 2, wherein the pharmaceutically acceptable carrier further includes a surfactant.

4. The composition of claim 3, wherein the surfactant is selected from L-1220, L-4370, or a combination thereof.

5. A method of treating a subject in need of homeostasis by administering to a subject in need of such treatment, a synergistic, homeostatic amount of a composition comprising one or more vitamins combined with one or more additive components selected from the group consisting of natural herbal extracts, and nature-identical synthetic chemical entities whose structures are active toward restoring homeostasis.

6. The method of claim 5, wherein the composition further includes a pharmaceutically acceptable carrier.

7. The method of claim 6, wherein the pharmaceutically acceptable carrier further includes a surfactant.

8. The method of claim 7, wherein the surfactant is selected from L-1220, L-4370, or a combination thereof.

9. The method of claim 5, where the synergistic composition further includes an OTC Drug and/or an Rx Drug ingredient, thereby providing synergy and improved homeostatic performance of the OTC or Prescription Drug.

10. The method of claim 9, wherein the OTC or Rx Drug ingredient is selected from the Approved Group listed in FDA 21 CFR.

11. The method of claim 5, wherein the homeostatic malfunction condition is caused by or related to a body condition selected from the group consisting of: Chronic and Acute Pain of the muscles, joints and ligaments, Migraine Headaches, Hair Loss, Thin, fragile skin, transparency and surface flaws which accompany Ageing, Alzheimer's disease, Diabetes, Arteriosclerosis, Actinic keratosis, Radiation Dermatitis, Pruritus, Insect bites, Anaphylactic Shock from serious allergic responses to allergens from Insect Bites, Food Allergies, Pollens and other Environmentally induced allergies, Carbuncles, Dermal burns due to exposure to sun, x-rays and/or excessive heat, Cancer, Mucositis, Aphthous ulcers, Periodontal and Gingivitis disease, Viral infections, Yeast and/or Microbial infections, Psoriasis, Crohn's disease, Lupus, Irritable Bowel disease, other Autoimmune Diseases and Diseases causally related to autoimmune diseases like fibromyalgia, Bruises, Wound Healing and Scar Formation (whether accidental or surgical), Rheumatoid and Osteoarthritis, multiple intensities of Dry Mouth or Xerostomia, and Dry Eyes.

12. The method of claim 5, wherein said composition is administered to the subject by a route selected from the group consisting of mucosal, dermal, oral, inhalation, injection, anal, nasal and other common routes known in the medical arts.

13. The method of claim 5, wherein the synergistic composition further includes one or more herbal extract and/or nature-identical synthetic alkylamides effective for achieving homeostasis.

14. The method of claim 13, where the alkylamides are selected from the group consisting of spilanthol, pellitorine, alpha-hydroxysanshool, and mixtures thereof

15. The method of claim 5, wherein the herbal extracts and/or nature-identical synthetic chemical entities are selected from the group consisting of: Corydalis yanhusuo, Scopolia carniolica, Asperula odorata, Salix alba, Aloysia citrodora, Rhodiola rosea, Raphanus sativus, Pogostemon patchouli, Paeonia alba, Thymus mastichina, Magnolia biondii, Ligusticum chuanxiong, Lavandula angustifolia, Cypripedium reginae, Ginko biloba, Gelsemium sempervirens, Tenacetum parthenium, Piscidia erythrina, Harpagophytum procumbens, Salvia sclerea, Nepta cataria, Petasites hybridus, Iris versicolor, Stachys betonica, Melissa officinalis, Ruta graveolens, Juniperus sabina, Hypericum perforatum, Anemone sylvestris, Pulsatilla vulgaris, Ranunculus gramineus, Delphinium staphisagria, Thuja occidentialis, Gaultheria procumbens, Pinus sylvestris, Mentha piperita, Schinus molle, Piper nigrum, Anacyclus pyrethrum, Urtica dioica, Moringa oleifera, Hyoscyamus niger, Conium maculatum, Allium sativum, Abies alba, Angelica sinensis, Croton tiglium, Juniperus virginiana, Capsicum annuum, Cinnamomum camphora, Acorns calamus, Melaleuca cajeputi, Dorema ammoniacum, Aconitum napellus, Illicium verum, Ferula foetida, Elettaria cardamomum, Sygyzium aromaticum, Trigonella foenum-graecum, Zingiber officinale, Solidago virgaurea, Armoracia rusticana, Glycyrrhiza glabra, Brassica nigra, Myristica fragrans, Allium sepa, Origanum vulgare, Zanthoxylum clava-herculis, Santalum album, Ulmas rubra, Eriodictyon californicum, Aerva lanata, Prunus serotine, Juniperus communis, Guaiacum officinale, Salvia officinalis, Malpighia glabra, Matricaria recutita, Eucalyptus globulus, Conyza Canadensis, Polygonum aviculare, Malva sylvestris, Aloe barbadensis, Origanum majorana, Sorbus Americana, Azadirachta indaca, Spilanthes acmella, Juglans regia, Melaluca alternifolia, Xanthorhiza simplicissima, Withania somnifera, Atropa belladonna, Valeriana officinalis, Morella cerifera, Syzygium cumini, Hypericum perforatum, Hamamelis virginiana, Rubrus fruticosus, Juglans cinerea, Bellis perennis, Gardenia augusta, Solidago virgaurea, Cetraria islandica, Avena sativa, Vinca minor, Santalum album, Polygonatum odoratum, Rhus aromatica, Vaccinium myrtillus, Trifolium pratense, Artium Lappa, Argentina anserina, Astragalus membranaceus, Paeonia lactaflfora, Baccharis trimera, Sanguinaria canadensis, Calendula officinalis, Murraya koenigii, Rumex crispus, Boswellia sacra, Baptisia tinctoria, Dryas octapetala, Parietaria officinalis, Arnica montana, Tanacetum vulgare, Echinacea angustifolia, Melilotus officinalis, Nymphaea alba, Aristolochia serpentaria, Hepatica nobilis, Rosmarinus officinalis, Symplocarpus foetidus, Gelsemium sempervirens, Phytolacca Americana, Staphania tetrandra, Melissa officinalis and Sterculia urens.

16. The method of claim 5, wherein the composition is administered to the subject prior to, immediately after, and/or long-after the homeostatic malfunction condition develops.

17. The method of claim 5, wherein the vitamin is selected from the group consisting of vitamins A, B, C, D, E, their pro-vitamin precursors, and mixtures thereof.

18. The method of claim 5, wherein the inflammatory conditions are controlled by affecting one or more biochemical control systems selected from the group consisting of Annexin A1, NRF2, NFkB, PI3K, AKT, MAPK, JNK, PIP2, PIP3, ERK1/2, cAMP, Adenylyl cyclase, TRPV1, TRPA1, TRPM8, PGE2, IL-1b, CXCR4 and CXCL 12.

19. The method of claim 5, wherein the inflammatory condition is reduced and homeostasis is restored by marshalling or enhancing the activity of the stem cells resident in the mammal by one or more of the stem cells' primary functions, (a) cell renewal and reproduction, (b) expressing chemotaxis allowing the stem cells to move toward a signal indicating an area of inflammatory condition, (c) cellular differentiation to produce the type of cell needed for reconstruction and (d) by reducing TNFa and increasing IL10, thus allowing a return to homeostasis.

20. The method of claim 17, wherein the vitamin is selected from D3 and pro-vitamin B5.
